(19) United States
(12) Patent Application Publication
(10) Pub. No.: US 2021/0121686 A1
(43) Pub. Date: Apr. 29, 2021

(54) MICROCURRENT SINUS TREATMENT DEVICE

(71) Applicant: TIVIC HEALTH SYSTEMS INC., MENLO PARK, CA (US)

(72) Inventors: JOHN CLAUDE, REDWOOD CITY, CA (US); JENNIFER ERNST, UNION CITY, CA (US); CHANDRASEKHAR DURISETY, FREMONT, CA (US); BLAKE TAYLOR GURFEIN, OAKLAND, CA (US)

(21) Appl. No.: 16/983,592

(22) Filed: Aug. 3, 2020

Related U.S. Application Data
(60) Provisional application No. 62/926,185, filed on Oct. 25, 2019.

(51) Int. Cl. A61N 1/05; A61N 1/18

(52) U.S. Cl. CPC A61N 1/0546; A61N 1/18

(57) ABSTRACT

According to an embodiment, a microcurrent sinus treatment device includes a hand holdable case configured to substantially contain active portions of a circuit for applying a sequence of voltage pulses to nerve nodes near the sinuses of a user. The hand holdable case includes a forward end terminating in a therapeutic electrode, and a return electrode comprising a portion of or disposed on a surface of the hand holdable case. The hand holdable case includes a dielectric spacer disposed between the therapeutic electrode and the return electrode, and a rearward portion of the hand holdable case terminating at an end a distance less than about four inches from the therapeutic electrode tip. The dielectric spacer and the return electrode may form a tapered surface narrowing toward the therapeutic electrode from a point of maximum girth disposed between the forward end and the rearward end of the hand holdable case.

### CROSS REFERENCE TO RELATED APPLICATIONS

The present application claims priority benefit from co-pending U.S. Provisional Patent Application No. 62/926,185, entitled “MICROCURRENT SINUS TREATMENT DEVICE,” filed Oct. 25, 2019 (docket number 3048-033-02), which, to the extent not inconsistent with the disclosure herein, is incorporated by reference.

### SUMMARY

According to an embodiment, a microcurrent sinus treatment device includes a circuit configured to deliver a sequence of voltage pulses carrying a therapeutic current, and a therapeutic electrode operatively coupled to the circuit. The therapeutic electrode may be configured to apply the sequence of voltage pulses to a user's skin surface adjacent to one of a plurality of nerve nodes subjacent to the user's skin surface. The therapeutic electrode may be in electrical continuity with a therapeutic current output node of the circuit. The microcurrent sinus treatment device includes a hand holdable case configured to substantially contain active portions of the circuit. The hand holdable case includes a forward end terminating in the therapeutic electrode, and a return electrode comprising a portion of or disposed on a surface of the hand holdable case. The return electrode may be in electrical continuity with a current return node of the circuit. The hand holdable case also includes a dielectric spacer disposed between the therapeutic electrode and the return electrode, and a rearward portion of the hand holdable case terminating at an end a distance less than about four inches from the therapeutic electrode tip. The dielectric spacer and the return electrode may form a tapered surface narrowing toward the therapeutic electrode from a point of maximum girth disposed between the forward end and the rearward end of the hand holdable case.

### BRIEF DESCRIPTION OF THE DRAWINGS

FIG. 1 is a perspective view of a microcurrent sinus treatment device showing a top and a side of the microcurrent sinus treatment device, according to an embodiment.

FIG. 2 is an exploded view of the microcurrent sinus treatment device of FIG. 1, according to an embodiment.

FIG. 3 is a perspective view of the microcurrent sinus treatment device of FIGS. 1 and 2, showing a back and side view of the microcurrent sinus treatment device, according to an embodiment.

FIG. 4 shows several views of a microcurrent sinus treatment device, according to another embodiment.

FIG. 5 shows dimensions, materials, and finish of a therapeutic electrode, according to an embodiment.

### DETAILED DESCRIPTION

In the following detailed description, reference is made to the accompanying drawings, which form a part hereof. In the drawings, similar symbols typically identify similar components, unless context dictates otherwise. Other embodiments may be used and/or other changes may be made without departing from the spirit or scope of the disclosure.

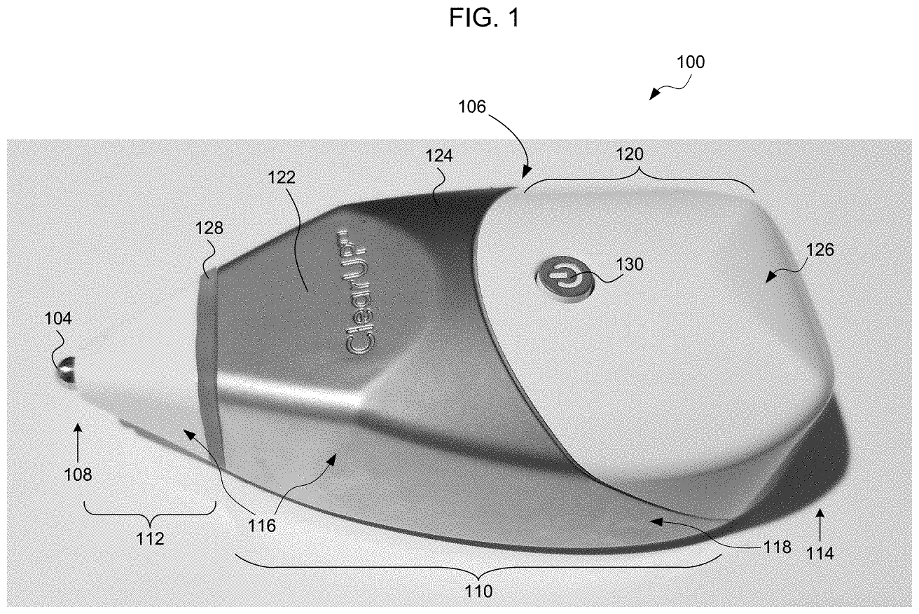

FIG. 1 is a perspective view of a microcurrent sinus treatment device showing a top and side of the sinus treatment device 100, according to an embodiment.

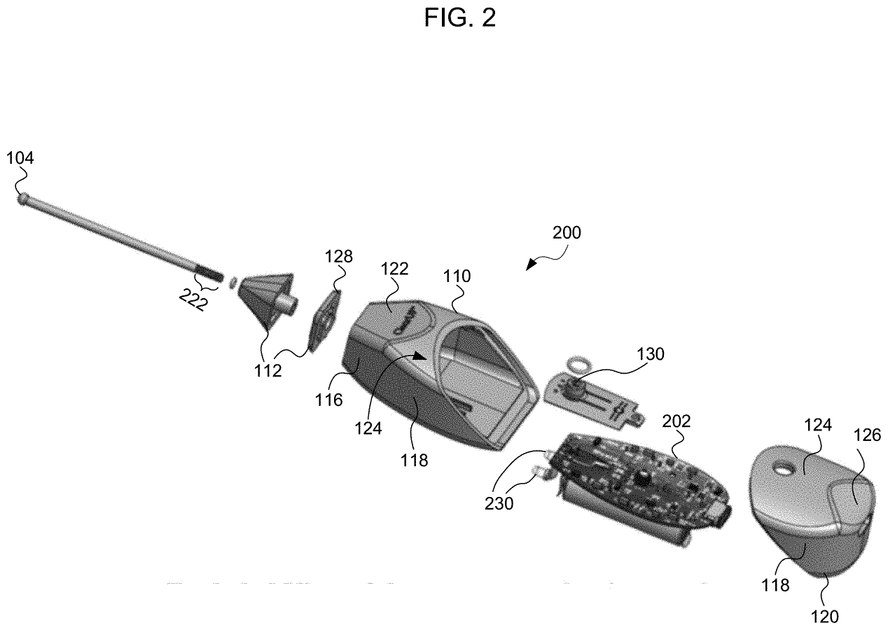

FIG. 2 is an exploded view 200 of the microcurrent sinus treatment device 100 of FIG. 1, according to an embodiment.

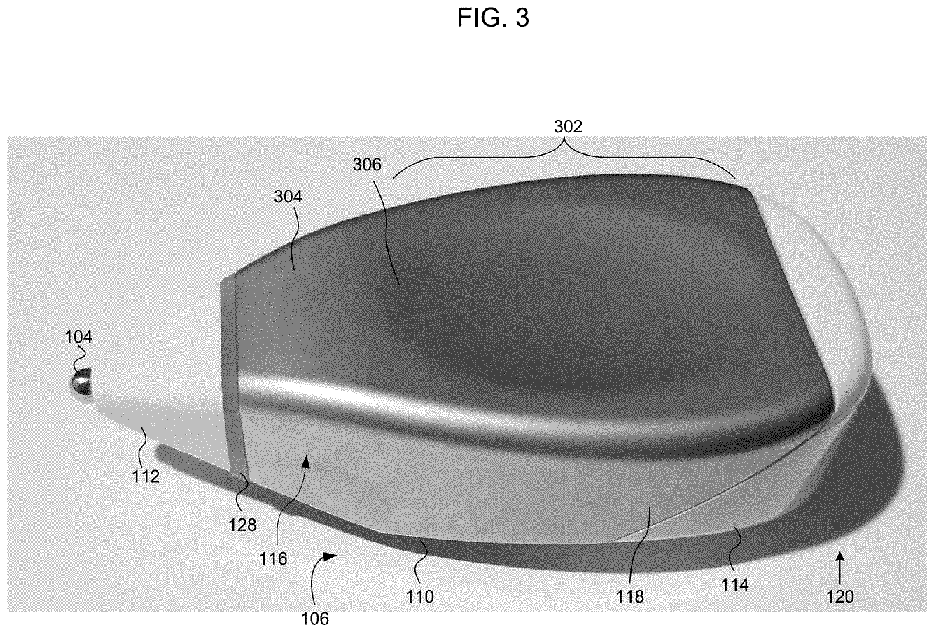

FIG. 3 is a perspective view of the microcurrent sinus treatment device 100, 200 of FIGS. 1 and 2, showing a back and side view of the microcurrent sinus treatment device 100, 200, according to an embodiment.

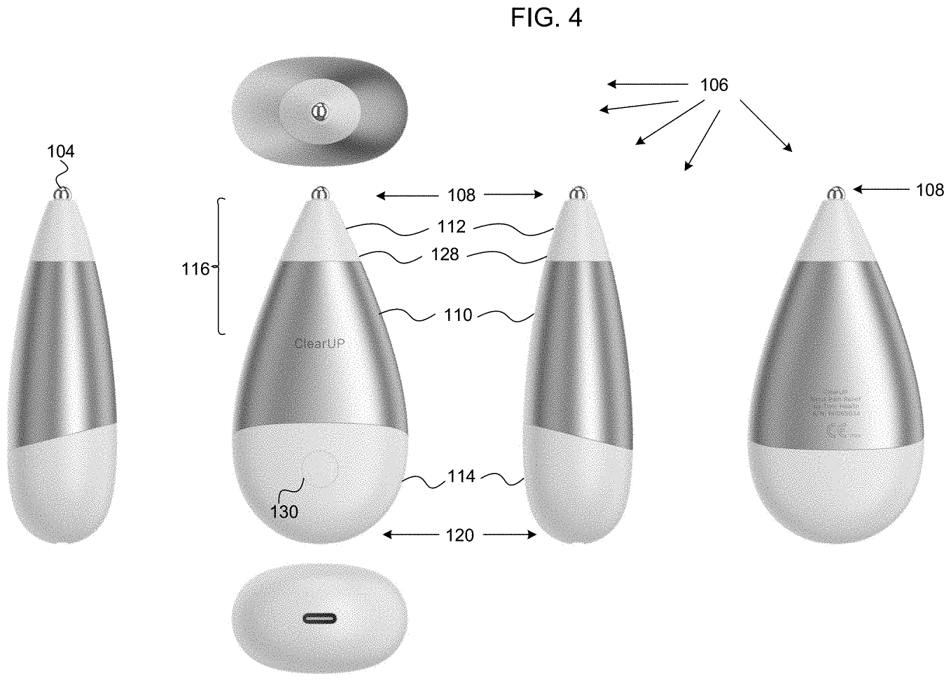

FIG. 4 shows several views of a microcurrent sinus treatment device, according to another embodiment.

The inventors note that the device described herein includes several features intended to provide a positive user experience.

With respect to structure, a therapeutic tip may include a screw thread to hold a case together with no external screws. According to an embodiment, a device includes a metal or metal-coated plastic return electrode on a case structure portion that occupies an area selected to make it nearly impossible for a user to hold the device without completing a circuit. In an embodiment, the device uses metal-plated plastic. In an embodiment, the device uses punched aluminum for an exposed portion of the return electrode.

Referring to FIGS. 1-3, according to an embodiment, a microcurrent sinus treatment device 100, 200 includes a circuit 202 configured to deliver a sequence of voltage pulses carrying a therapeutic current, and a therapeutic electrode 104 operatively coupled to the circuit 202. In an embodiment, the therapeutic electrode 104 may be configured to apply the sequence of voltage pulses to a user's skin surface adjacent to a region of relatively low electrical impedance through the user's skin and to a return electrode on a surface of the microcurrent sinus treatment device 100, 200. According to embodiments, the region of relatively low electrical impedance corresponds to one of a plurality of nerve nodes subjacent to the user's skin surface. In an embodiment, the therapeutic electrode 104 may be in electrical continuity with a therapeutic current output node of the circuit 202. According to an embodiment, the microcurrent sinus treatment device 100, 200 includes a hand holdable case 106 configured to substantially contain active portions of the circuit 202. In an embodiment, the hand holdable case 106 includes a forward end 108 terminating in the therapeutic electrode 104, and a return electrode 110 comprising a portion of or disposed on a surface of the hand holdable case 106. In an embodiment, the return electrode 110 may be in electrical continuity with a current return node of the circuit 202. In an embodiment, the hand holdable case 106 includes a dielectric spacer 112 disposed between the therapeutic electrode 104 and the return electrode 110, and a rearward portion 114 of the hand holdable case 106 terminating at an end 120 a distance selected to comfortably fit in an adult human hand, typically less than about four inches from the therapeutic electrode 104 tip. In an embodiment, the dielectric spacer 112 and the return electrode 110 form a tapered surface 116 narrowing toward the therapeutic electrode 104 from a point of maximum girth 118 disposed between the forward end 108 and the rearward end 120 of the hand holdable case 106.

According to an embodiment, the therapeutic electrode 104 is configured to clamp the return electrode 110 against the rearward portion 114 of the hand holdable case 106 to hold the dielectric spacer 112 and the return electrode 110 together with the rearward portion 114 of the hand holdable case 106. In an embodiment, the therapeutic electrode 104 includes a threaded portion 222 configured to screw into a hole (not shown) formed inside the rearward portion 114 of the hand holdable case 106. In an embodiment, the placement of the therapeutic electrode 104, the dielectric spacer 112, and the return electrode 110 are configured to cause the user's body to complete a circuit between the therapeutic electrode 104 and the return electrode 110.

In an embodiment, the tapered case is adaptable to a large range of hand sizes. In an embodiment, the rearward end 120 of the hand holdable case 106 is less than three inches from the therapeutic electrode 104 tip. In an embodiment, the tapered surface 116 is conducive to cause the user's hand to naturally contact the return electrode 110. In an embodiment, the tapered surface 116 is conducive to provide satisfactory control for holding the therapeutic electrode 104 against the user's skin superjacent to each of the plurality of nerve nodes.

According to an embodiment, in FIGS. 1-3, the hand holdable case 106 forms a surface having an indentation 302 larger than an average user's thumb on a back side 304 of the hand holdable case 106 such that a front portion 306 of the indentation 302 extends toward the therapeutic electrode 104 and away from the point of maximum girth 118 of the hand holdable case 106. In one embodiment, the indentation 302 is disposed with at least a majority of its area between the therapeutic electrode 104 and the point of maximum girth 118. In another embodiment, the indentation 302 is disposed partially extending beyond the point of maximum girth 118 toward the rearward end 120 of the hand holdable case 106.

According to an embodiment, the dielectric spacer 112 defines a concave insulated surface near the therapeutic electrode 104 to make clearance for the user's cheek and/or nose.

According to an embodiment, the hand holdable case 106 defines a tapered surface 122 on a top 124, between the point of maximum girth 118 and the forward end 108, configured to provide a finger hold. In an embodiment, the tapered surface 122 forms a facet relative to other portions of the top 124. According to an embodiment, the hand holdable case 106 defines a convex curved surface 126, on the top 124 between the point of maximum girth 118 and the rearward end 120, configured to fit into a hollow of the user's palm.

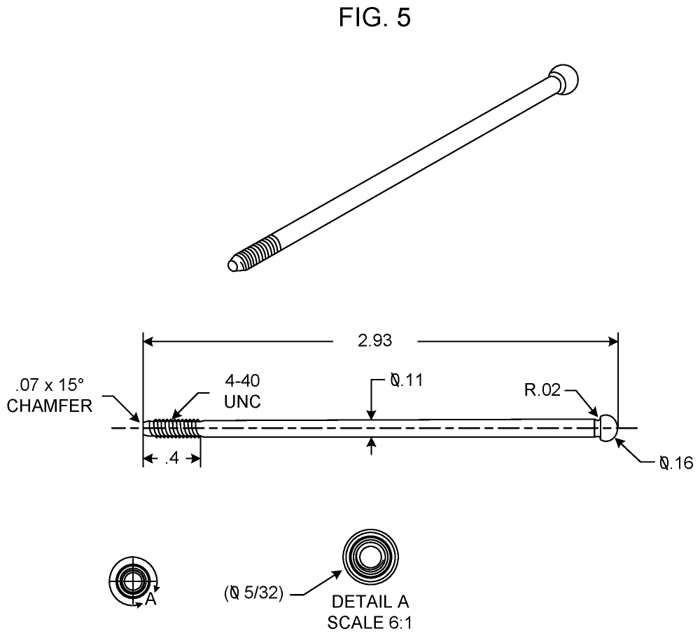

According to a embodiment, the hand holdable case 106 defines a tapered side 116 to accommodate finger placement. According to an embodiment, the hand holdable case 106 further includes a light pipe 128 disposed between the forward end 108 and the return electrode 110, the light pipe 128 being configured to output an illumination indicator from a light emitting diode (LED) 230 disposed on the circuit 202 to indicate an operating condition to the user. According to an embodiment, the microcurrent sinus treatment device 100, 200 further includes a button 130 configured to cause the circuit 202 to enter a low current, nerve node finding mode, the circuit 202 being further configured to enter a high current therapeutic voltage pulse mode when a nerve node is found and to automatically shut off when a dose of therapeutic voltage pulses have been delivered. According to an embodiment, the therapeutic electrode 104 may be configured to maximize comfort for the user. This may be accomplished by keeping a diameter and radius of the therapeutic electrode 104 tip at the forward end 108 of the hand holdable case 106 large enough to avoid applying undue pressure against the user's skin. The inventors have found that maximizing the diameter and radius for comfort should be balanced against localization of current flow across the user's skin. In an embodiment, an exposed portion of the therapeutic electrode 104 may have a diameter greater than or equal to 1/16 of an inch (0.0625″) ( 1/32″=0.031″ radius) and less than ¼″ (0.25″) diameter (⅛″ =0.125″ radius). In another embodiment, the exposed portion of the therapeutic electrode 104 may have a diameter greater than or equal to 3/32″ (=0.094″) ( 3/64″=0.047″) and less than or equal to 3/16″ (0.188″) diameter and 3/32″ =0.094″ radius. In another embodiment, the exposed portion of the therapeutic electrode 104 may have a diameter equal to about 5/32″ (0.16″) and 5/64″ (0.078″ radius). Relevant dimensions, according to an embodiment, are shown in FIG. 5.

According to embodiments, the inventors have noted three primary ways users hold the device:

1. Thumb directed—the user puts his/her thumb in the indentation 302 and points toward the user's face. The user's fingers wrap around the hand holdable case 106.

2. Finger directed—the user places his/her index finger on the tapered surface 116 and points toward the user's face. The user's fingers and thumb wrap around the hand holdable case 106.

3. Hybrid—the user places his/her thumb on the indentation 302 and places his/her index finger on the side, both thumb and index finger point toward the user's face. The user's middle finger stabilizes along the front. (Some users were found to be unable to use this technique.)

All three approaches are amenable to right-handed or left-handed use. Users naturally fell into one of the three grips. Features on the hand holdable case 106 enable one or more of the grips.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A microcurrent sinus treatment device, comprising;
a circuit configured to deliver a sequence of voltage pulses carrying a therapeutic current;
a therapeutic electrode operatively coupled to the circuit, the therapeutic electrode being configured to apply the sequence of voltage pulses to a user's skin surface adjacent to a region of relatively low electrical impedance through the user's skin, the therapeutic electrode being in electrical continuity with a therapeutic current output node of the circuit; and
a hand holdable case configured to substantially contain active portions of the circuit, the hand holdable case including:
a forward end terminating in the therapeutic electrode;
a return electrode comprising a portion of or disposed on a surface of the hand holdable case, the return electrode being in electrical continuity with a current return node of the circuit;
a dielectric spacer disposed between the therapeutic electrode and the return electrode; and
a rearward portion of the hand holdable case terminating at an end a distance less than about four inches from the therapeutic electrode tip;
wherein the dielectric spacer and the return electrode form a tapered surface narrowing toward the therapeutic electrode from a point of maximum girth disposed between the forward end and the rearward end of the hand holdable case.

2. The microcurrent sinus treatment device of claim 1, wherein the region of relatively low electrical impedance through the user's skin corresponds to a location superjacent to one of a plurality of nerve nodes subjacent to the user's skin surface

3. The microcurrent sinus treatment device of claim 1, wherein the therapeutic electrode is configured to clamp the return electrode against the rearward portion of the hand holdable case to hold the dielectric spacer and the return electrode together with the rearward portion of the hand holdable case.

4. The microcurrent sinus treatment device of claim 3, wherein the therapeutic electrode includes a threaded portion configured to screw into a hole formed inside the rearward portion of the hand holdable case.

5. The microcurrent sinus treatment device of claim 1, wherein the placement of the therapeutic electrode, the dielectric spacer, and the return electrode are configured to cause the user's body to complete a circuit between the therapeutic electrode and the return electrode.

6. The microcurrent sinus treatment device of claim 1, wherein the rearward end of the hand holdable case is less than three inches from the therapeutic electrode tip.

7. The microcurrent sinus treatment device of claim 1, wherein the hand holdable case forms a surface having an indentation larger than an average user's thumb on a back side of the hand holdable case such that a front portion of the indentation extends toward the therapeutic electrode and away from the point of maximum girth of the hand holdable case.

8. The microcurrent sinus treatment device of claim 7, wherein the indentation is disposed with at least a majority of its area between the therapeutic electrode and the point of maximum girth.

9. The microcurrent sinus treatment device of claim 1, wherein the dielectric spacer defines a concave insulated surface near the therapeutic electrode to make clearance for the user's cheek and/or nose.

10. The microcurrent sinus treatment device of claim 1, wherein the hand holdable case defines a tapered surface on a top, between the point of maximum girth and the forward end, configured to provide a finger hold.

11. The microcurrent sinus treatment device of claim 10, wherein the tapered surface forms a facet relative to other portions of the top.

12. The microcurrent sinus treatment device of claim 1, wherein the hand holdable case defines a convex curved surface, on the top between the point of maximum girth and the rearward end, configured to fit into a hollow of the user's palm.

13. The microcurrent sinus treatment device of claim 1, wherein the hand holdable case defines a tapered side to accommodate finger placement.

14. The microcurrent sinus treatment device of claim 1, wherein the hand holdable case further comprises a light pipe disposed between the forward end and the return electrode, the light pipe being configured to output an illumination indicator from a light emitting diode (LED) disposed on the circuit to indicate an operating condition to the user.

15. The microcurrent sinus treatment device of claim 1, further comprising a button configured to cause the circuit to enter a low current, nerve node finding mode, the circuit being further configured to enter a high current therapeutic voltage pulse mode when a nerve node is found and to automatically shut off when a dose of therapeutic voltage pulses have been delivered.

16. The microcurrent sinus treatment device of claim 1, wherein an exposed portion of the therapeutic electrode has a radius of curvature of greater than or equal to 0.031 inch and less than or equal to 0.25 inch.

17. The microcurrent sinus treatment device of claim 16, wherein the exposed portion of the therapeutic electrode has a radius of curvature greater than 0.047 inch and less than 0.094 inch.

18. The microcurrent sinus treatment device of claim 17, wherein the exposed portion of the therapeutic electrode has a radius of curvature of about 0.08 inch.

19. The microcurrent sinus treatment device of claim 1, wherein the exposed portion of the therapeutic electrode is formed from stainless steel.
